(19) United States
(12) Patent Application Publication
(10) Pub. No.: US 2006/0191043 A1
(43) Pub. Date: Aug. 24, 2006

(54) Low maintenance turfgrass

(76) Inventors: Shirley Guo, Chesterfield, MO (US); Lisa Lee, Marysville, OH (US); Robert W. Harriman, Delaware, OH (US); Gregory Heck, Crystal Lake Park, MO (US); Rebecca Torisky, Waterloo, NY (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 11/409,365

(22) Filed: Apr. 20, 2006

Related U.S. Application Data
(62) Division of application No. 11/060,448, filed on Feb. 17, 2005, now abandoned.
(60) Provisional application No. 60/545,026, filed on Feb. 17, 2004.

(51) Int. Cl. C12N 15/82; A01H 5/00; A01H 1/00

(52) U.S. Cl. 800/287; 800/320

(57) ABSTRACT

The invention relates to DNA constructs and methods for producing transgenic glyphosate tolerant, dwarf turfgrass plants that contain these constructs. The invention also relates to the maintenance of the transgenic turfgrass stand.

This application claims benefit under 35USC §119(e) of U.S. provisional application Ser. No. 60/54,5026 filed Feb. 17, 2004, herein incorporated by reference in its entirety.

### FIELD OF THE INVENTION

The present invention relates to the field of plant molecular biology. More specifically, the invention relates to DNA constructs and methods for producing transgenic glyphosate tolerant, dwarf turfgrass plants that contain these constructs. The invention also relates to the maintenance of the transgenic turfgrass stand in a lawn.

### BACKGROUND OF THE INVENTION

Turfgrass is an important plant grown all over the world. The maintenance of a turfgrass lawn can be expensive and time consuming, and the control of weeds in a lawn is particularly problematic. Annual grasses, such as, crabgrass, foxtail, dallisgrass, and goosegrass must be controlled by use of a variety of herbicides including bensulide, dithiopyr, oxadiazon, fenoxaprop and prodiamine applied at specific rates, environmental conditions, and seasons by expert applicators in order to be effective. Annual and perennial broadleaf weeds may be controlled in a lawn by applications of herbicides that include 2,4-D, MCPP, dicamba, and mixtures of these. There is a need for a glyphosate tolerant turfgrass to replace the use of these herbicides and to provide a method for effective grass and broadleaf weed control in a lawn. Plant biotechnology has demonstrated the methods necessary to introduce herbicide tolerance in many plant species. In particular, genetically engineered tolerance to glyphosate herbicide has been applied to many crop species. Glyphosate is a broad spectrum, environmentally friendly herbicide, it would be desirable to have turfgrass species that are tolerant to glyphosate herbicide.

Mechanical mowing of turfgrass is a time consuming and expensive activity, and the gasoline powered mowing equipment can contribute to air quality issues, particularly in urban areas. Homeowners and businesses would benefit from reduced mowing expenses and time savings. The use of a dwarf turfgrass would result in less mowing, which would be advantageous for most lawns. Dwarf grasses have another advantage; they are not as invasive into unwanted areas as other non-dwarf varieties. However, because of this trait, dwarf turfgrasses often do not perform well because they are slow to establish in a lawn and once established cannot compete effectively with weeds. There is a need in the turfgrass industry for a dwarf turfgrass where growth can be induced when needed and is also herbicide tolerant. Turfgrasses, such as, creeping bentgrass (Agrostis stolonifera), St Augustinegrass (Stenotaphrum secundatum) and Kentucky bluegrass (Poa pratensis) are important turfgrass species for lawns, playing fields, and golf courses. Genetically engineered herbicide tolerant, dwarf phenotypes into grasses such as these would have lower maintenance costs due to the use of a single herbicide to control most major weed problems and to reduced mowing expenses.

N-phosphonomethylglycine, also known as glyphosate, is a well-known herbicide that has activity on a broad spectrum of plant species. Glyphosate is the active ingredient of Roundup® (Monsanto Co., St Louis, Mo.), a safe herbicide having a desirably short half-life in the environment. When applied to a plant surface, glyphosate moves systemically through the plant. Glyphosate is phytotoxic due to its inhibition of the shikimic acid pathway, which provides a precursor for the synthesis of aromatic amino acids. Glyphosate inhibits the enzyme 5-enolpyruvyl-3-phosphoshikimate synthase (EPSPS) found in plants. Glyphosate tolerance can also be achieved by the expression of bacterial EPSPS variants and plant EPSPS variants that have lower affinity for glyphosate and therefore retain their catalytic activity in the presence of glyphosate, for example, U.S. Pat. Nos. 5,633,435; 5,094,945, 4,535,060, 6,040,497, and WO04/07443, herein incorporated by reference in their entirely.

Degradation of bioactive gibberellic acid (GA) in plant tissues affects plant cell elongation and results in a dwarf plant phenotype. Genes from Arabidopsis and Phaseolus coccineus have been identified that encode for enzymes that have gibberellin 2-oxidase activity (U.S. Pat. No. 6,670,527 and Thomas, et al., Proc. Natl. Acad. Sci. U.S.A. 96: 4698-4703, 1999). Other GA 2-oxidase coding sequences have also been isolated from various plant species that include cotton, soybean, maize and rice (US Patent pub 20030233679, Sakai et al., J Plant Res. 116:161-164, 2003). The GA 2-oxidase gene product functions by controlling bioactive gibberellin levels. Hydroxylation of bioactive GAs, such as GA₁ and GA₄, by GA 2-oxidase renders them inactive, while hydroxylation of biosynthetic precursors, such as GA₉ and GA₂₀, creates non-preferable substrates for GA biosynthetic enzymes. Overexpression of the GA 2-oxidase protein can therefore be used to directly inactivate GA levels or indirectly down-regulate endogenous bioactive GA levels by affecting the substrate levels. To restore cell elongation, plants can be treated exogenously with bioactive GA₃ or GA analogs that are not substrates for 2-oxidase. Gibberellic acid induces bolting of flowering structures in many plants. The bolting response can be largely prevented by reducing the levels of GA in the plant. This is a particularly useful trait in a low maintenance lawn as bolting is unsightly in a lawn and would require mowing.

The present invention relates to a method to provide transgenic herbicide tolerant, dwarf turfgrass species, to the transgene DNA compositions contained therein, and to methods for maintaining a turfgrass stand comprising the transgenic turfgrass.

### SUMMARY OF THE INVENTION

The invention is generally related to a method for providing a transgenic turfgrass plant that is both herbicide tolerant and dwarf. The method describes a DNA construct that comprises a herbicide tolerance gene and a dwarfing gene that is transformed into a recipient turfgrass cell and incorporated into the genome of the cell, the cell is then regenerated into a turfgrass plant. The transgenic turfgrass plant is tolerant to at least one herbicide and has a reduced growth phenotype. The invention more specifically describes a herbicide tolerance gene that provides tolerance to glyphosate and a dwarfing gene that is a gibberellic acid level reducing gene, such as, a GA 2-oxidase gene.

In another aspect of the invention there is provided a turfgrass plant and progeny thereof that contain a DNA construct comprising a herbicide tolerance gene and a gibberellic acid level reducing gene, wherein the plant is herbicide tolerant and dwarf. The turfgrass plant comprises any turfgrass species useful as a lawn, golf course, sports field or other commercial and noncommercial use, the turfgrass includes, but is not limited to bentgrass, bluegrass, and St Augustinegrass.

In another aspect of the invention, there is provided a turfgrass stand comprising a transgenic turfgrass that is glyphosate tolerant and dwarf, wherein the growth phenotype can be controlled by exogenous application of gibberellin containing formulations and weeds can be controlled by exogenous application of glyphosate formulations.

### BRIEF DESCRIPTION OF THE DRAWINGS

FIG. 1. DNA construct map of pMON39073

FIG. 2. DNA construct map of pMON70598

FIG. 3. DNA construct map of pMON39078

FIG. 4. DNA construct map of pMON39081

FIG. 5. DNA construct map of pMON39083

### DETAILED DESCRIPTION OF THE PREFERRED EMBODIMENTS

The present invention relates to transgenic herbicide tolerant, dwarf turfgrass created by transformation with a DNA construct that comprises a herbicide tolerance gene and a dwarfing gene. The transgenic turfgrass will require less frequent mowing and weed control can be achieved by treatment with a herbicide for which the turfgrass is tolerant. Additional traits of the turfgrass include inducible growth and reduced bolting or flowering. As used herein, the term “turfgrass” means any grass species cultivated in a lawn, golf course, sports field, or other areas that comprise a turfgrass stand and includes all plant varieties that can be bred with turfgrasses. Examples of turfgrasses include: bahiagrass, bentgrass, bermudagrass, bluegrass, buffalograss, carpetgrass, centipedegrass, fescue, paspalum, ryegrass, St Augustinegrass, wheatgrass, and zoysia.

Herbicides for which transgenic plant tolerance has been demonstrated and the method of the present invention can be applied, include but are not limited to: glyphosate, glufosinate, sulfonylureas, imidazolinones, bromoxynil, delapon, cyclohezanedione, protoporphyrionogen oxidase inhibitors, and isoxaflutole herbicides. Polynucleotide molecules encoding proteins involved in herbicide tolerance are known in the art, and include, but are not limited to a polynucleotide molecule encoding 5-enolpyruvylshikimate-3-phosphate synthase (EPSPS, described in U.S. Pat. Nos. 5,627,061, 5,633,435, 6,040,497; Padgette et al. Herbicide Resistant Crops, Lewis Publishers, 53-85, 1996; and Penaloza-Vazquez, et al Plant Cell Reports 14:482-487, 1995; and aroA (U.S. Pat. No. 5,094,945) for glyphosate tolerance; bromoxynil nitrilase (Bxn) for bromoxynil tolerance (U.S. Pat. No. 4,810,648); phytoene desaturase (crtI, Misawa et al, (1993) Plant J. 4:833-840, and (1994) Plant J. 6:481489); for tolerance to norflurazon, acetohydroxyacid synthase (AHAS, aka ALS, Sathasiivan et al. Nucl. Acids Res. 18:2188-2193, 1990); and the bar gene for tolerance to glufosinate and bialaphos (DeBlock, et al. EMBO J. 6:2513-2519, 1987).

Through plant genetic engineering methods, it is possible to produce glyphosate tolerant plants by inserting into the plant genome a DNA molecule that causes the production of higher levels of wild-type EPSPS (Shah et al., Science 233:478-481, 1986). Glyphosate tolerance can also be achieved by the expression of EPSPS variants that have lower affinity for glyphosate and therefore retain their catalytic activity in the presence of glyphosate (U.S. Pat. No. 5,633,435). Enzymes that degrade glyphosate in the plant tissues (U.S. Pat. No. 5,463,175) are also capable of conferring cellular tolerance to glyphosate. Such genes, therefore, allow for the production of transgenic crops that are tolerant to glyphosate, thereby allowing glyphosate to be used for effective weed control with minimal concern of crop damage. For example, glyphosate tolerance has been genetically engineered into corn (U.S. Pat. Nos. 5,554,798; 6,040,497), wheat (Zhou et al. Plant Cell Rep. 15:159-163, 1995), soybean (WO 9200377) and canola (WO 9204449).

Variants of the wild-type EPSPS enzyme have been isolated that are glyphosate-resistant as a result of alterations in the EPSPS amino acid coding sequence (Kishore et al., Annu. Rev. Biochem. 57:627-663, 1988; Schulz et al., Arch. Microbiol. 137:121-123, 1984; Sost et al., FEBS Lett. 173:238-241, 1984; Kishore et al., In “Biotechnology for Crop Protection” ACS Symposium Series No. 379. eds. Hedlin et al., 37-48, 1988). These variants typically have a higher Kᵢ for glyphosate than the wild-type EPSPS enzyme that confers the glyphosate-tolerant phenotype, but these variants are also characterized by a high Kₘ for PEP that makes the enzyme kinetically less efficient. For example, the apparent Kₘ for PEP and the apparent Kᵢ for glyphosate for the native EPSPS from E. coli are 10 μM and 0.5 μM while for a glyphosate-resistant isolate having a single amino acid substitution of an alanine for the glycine at position 96 these values are 220 μM and 4.0 mM, respectively. U.S. Pat. No. 6,040,497 reports that the mutation known as the TIPS mutation (a substitution of isoleucine for threonine at amino acid position 102 and a substitution of serine for proline at amino acid position 106) comprises two mutations that when introduced into the polypeptide sequence of Zea mays EPSPS confers glyphosate resistance to the enzyme. Transgenic plants containing this mutant enzyme are tolerant to glyphosate. Identical mutations may be made in glyphosate sensitive EPSPS enzymes from other plant sources to create glyphosate resistant enzymes. These glyphosate resistant enzymes can be used in the present invention.

“Glyphosate” refers to N-phosphonomethylglycine and its salts, glyphosate is the active ingredient of Roundup® herbicide (Monsanto Co. St Louis, Mo.). Treatments with “glyphosate herbicide” refer to treatments with the Roundup®, Roundup Ultra®, Roundup Pro® herbicide or any other herbicide formulation containing glyphosate. Examples of commercial formulations of glyphosate include, without restriction, those sold by Monsanto Company as ROUNDUP®, ROUNDUP® ULTRA, ROUNDUP® ULTRAMAX, ROUNDUP® WeatherMAX ROUNDUP® CT, ROUNDUP® EXTRA, ROUNDUP® BIACTIVE, ROUNDUP® BIOFORCE, RODEO®, POLARIS®, SPARK® and ACCORD® herbicides, all of which contain glyphosate as its isopropylammonium salt; those sold by Monsanto Company as ROUNDUP® DRY and RIVAL® herbicides, which contain glyphosate as its ammonium salt; that sold by Monsanto Company as ROUNDUP® GEOFORCE, which contains glyphosate as its sodium salt; and that sold by Zeneca Limited as TOUCHDOWN® herbicide, which contains glyphosate as its trimethylsulfonium salt.

A plant dwarfing gene, for example, a gibberellic acid level reducing enzyme, such as, a GA 2-oxidase gene product that functions by controlling bioactive gibberellin levels can be used in the present invention (U.S. Pat. No. 6,670,527 and US Patent pub 20030233679). Hydroxylation of bioactive GAs, such as GA₁ and GA₄, by GA 2-oxidase renders them inactive, while hydroxylation of biosynthetic precursors, such as GA₉ and GA₂₀, creates non-preferable substrates for GA biosynthetic enzymes. Overexpression of the GA 2-oxidase protein can therefore be used to directly inactivate GA levels or indirectly down-regulate endogenous bioactive GA levels by affecting the substrate levels, thereby reducing internode and leaf elongation. To restore elongation capacity, the plants can be treated exogenously with bioactive gibberellic acid, such as, GA₃ or GA analogs that are not substrates for the GA 2-oxidase. Seeds and plants can also be treated with nonpreferred substrates or by treatment with excess amounts of preferred substrates.

Different GA 2-oxidase genes exist whose proteins have varied substrate specificities. The known GA 2-oxidase enzymes have different substrate preferences, catalytic properties, and tissue/developmental distributions. These differences in expression and catalytic capabilities may reflect mechanisms for the fine control of specific GAs and their relative contributions to regulating plant growth and development. Additional GA 2-oxidase genes may exist in higher plants genomes as evidenced by a wide variety of GA metabolites identified (Owen et al., Phytochemistry 97: 331-337, 1998). GA 2-oxidases isolated from different plant species (Arabidopsis and bean, U.S. Pat. No. 6,670,527; soybean, cotton, maize, and Arabidopsis US Patent pub 20030233679; rice, Sakai et al., J Plant Res. 116:161-164, 2003) can be used in the present invention to create dwarf turfgrass plants. Other methods and uses of polynucleic acid molecules described in US Patent pub 20030233679, such as, antisense to GA biosynthetic enzyme coding sequences and pathway diverting enzymes are also contemplated in the present invention. Additional dwarfing genes that can be used in the present invention, include but are not limited to, cytochrome P450-t (U.S. Pat. No. 5,952,545); BAS1 gene (U.S. Pat. No. 6,534,313); ro1 (A, B, and C) genes; phyA gene (U.S. Pat. No. 5,945,579); crtO gene (Harker and Hirschberg FEBS Lett. 404:129-134, 1997); lycopene cyclase gene; OsMADS45 gene and OsMADS1 gene. Dwarfing gene expression of the present invention can affect leaf length, stolon length, flower head height, flower formation, timing of bolting, and other growth and development phenotypes associated with cell elongation.

A DNA construct comprises a number of operably linked DNA molecules. One such element is a “promoter” or “promoter region” that refers to a polynucleic acid molecule that functions as a regulatory element, usually found upstream (5′) to a coding sequence, that controls expression of the coding sequence by controlling production of messenger RNA (mRNA), by providing the recognition site for RNA polymerase, and/or other factors necessary for start of transcription at the correct site. As contemplated herein, a promoter or promoter region includes variations of promoters derived by means of ligation to various regulatory sequences, random or controlled mutagenesis, and addition or duplication of enhancer sequences. The promoter region disclosed herein, and biologically functional equivalents thereof, are responsible for driving the transcription of coding sequences under their control when introduced into a host as part of a suitable recombinant DNA construct, as demonstrated by its ability to produce mRNA.

A variety of promoters specifically active in vegetative tissues, such as leaves, stems, roots and tubers, can be used to express the EPSPS polynucleic acid molecules and dwarfing genes of the present invention. Examples of leaf-specific promoters include, but are not limited to the ribulose biphosphate carboxylase (RbcS2 or RuBISCO) promoters (see, for example, Matsuoka et al., Plant J. 6:311-319, 1994); the light harvesting chlorophyll a/b binding protein gene promoter (see, for example, Shiina et al., Plant Physiol. 115:477-483, 1997; Casal et al., Plant Physiol. 116:1533-1538, 1998); the Arabidopsis thaliana myb-related gene promoter (Atmyb5) (Li et al., FEBS Lett. 379:117-121, 1996), and the Zea mays PPDK promoter (WO200119976A2). Constitutive viral promoters, such as, those derived from the figwort mosaic virus (U.S. Pat. Nos. 6,051,753 and 6,018,100) and cauliflower mosaic virus (U.S. Pat. Nos. 5,352,605 and 5,196,525) are useful in DNA constructs of the present invention as well as chimeric promoter molecules (U.S. Pat. No. 6,660,911) containing enhancer elements derived from these and other viral promoters.

Also another example of a useful promoter is that which controls the expression of knl-related genes from maize and other species that show meristem-specific expression (see, for example, Granger et al., Plant Mol. Biol. 31:373-378, 1996; Kerstetter et al., Plant Cell 6:1877-1887, 1994; Hake et al., Philos. Trans. R. Soc. Lond. B. Biol. Sci. 350:45-51, 1995). Another example of a meristematic promoter is the Arabidopsis thaliana KNAT1 promoter. In the shoot apex, KNAT1 transcript is localized primarily to the shoot apical meristem; the expression of KNATI in the shoot meristem decreases during the floral transition and is restricted to the cortex of the inflorescence stem (see, for example, Lincoln et al., Plant Cell 6:1859-1876, 1994).

It is recognized that additional promoters that may be utilized are described, for example, in U.S. Pat. Nos. 5,378,619; 5,391,725; 5,428,147; 5,447,858; 5,608,144, 5,614,399; 5,633,441; 5,633,435, and 4,633,436 to provide the desired herbicide tolerant and dwarf turfgrass phenotype described in the present invention. It is further recognized that the exact boundaries of regulatory sequences may not be completely defined, DNA fragments of different lengths may have identical promoter activity.

Introns (e.g., U.S. Pat. No. 5,424,412) are DNA regulatory elements that provide a splice site to facilitate expression of the gene, such as the maize Hsp70 intron (U.S. Pat. No. 5,593,874).

The translation leader sequence is a DNA molecule located between the promoter of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences include maize and petunia heat shock protein leaders (U.S. Pat. No. 5,362,865), plant virus coat protein leaders, plant rubisco gene leaders among others (Turner and Foster, Molecular Biotechnology 3:225, 1995).

The “3′ non-translated sequences” means DNA sequences located downstream of a structural polynucleotide sequence and include sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA. An example of the polyadenylation sequence is the nopaline synthase 3′ sequence (nos 3′; Fraley et al., Proc. Natl. Acad. Sci. USA 80: 4803-4807, 1983). The use of different 3′ non-translated sequences is exemplified by Ingelbrecht et al., Plant Cell 1:671-680, 1989.

The laboratory procedures in recombinant DNA technology used herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Molecular Cloning: A Laboratory Manual, 2nd ed., vol. 1-3, ed. Sambrook et al., Cold Spring Harbor Laboratory Press, Cold Spring Harbor, N.Y., 1989 (hereinafter, “Sambrook et al., 1989”); and Current Protocols in Molecular Biology, ed. Ausubel et al., Greene Publishing and Wiley-Interscience, New York, 1992 (with periodic updates) (hereinafter, “Ausubel et al., 1992”).

Methods of transformation of plant cells or tissues include, but are not limited to Agrobacterium mediated transformation method and the Biolistics or particle-gun mediated transformation method. Suitable plant transformation vectors for the purpose of Agrobacterium mediated transformation include those elements derived from a tumor inducing (Ti) plasmid of Agrobacterium tumefaciens, for example, right border (RB) regions and left border (LB) regions, and others disclosed by Herrera-Estrella et al., Nature 303:209 (1983); Bevan, Nucleic Acids Res.12:8711-8721 (1984); Klee et al., Bio-Technology 3(7):637-642 (1985). In addition to plant transformation vectors derived from the Ti or root-inducing (Ri) plasmids of Agrobacterium, alternative methods can be used to insert the DNA constructs of this invention into plant cells. Such methods may involve, but are not limited to, for example, the use of liposomes, electroporation, chemicals that increase free DNA uptake, free DNA delivery via microprojectile bombardment, and transformation using viruses or pollen. “Transformation” refers to a process of introducing an exogenous polynucleic acid molecule (for example, a DNA construct, a recombinant polynucleic acid molecule) into a cell or protoplast and that exogenous polynucleic acid molecule is incorporated into a host cell genome or an organelle genome (for example, chloroplast or mitochondria) or is capable of autonomous replication. “Transformed” or “transgenic” refers to a cell, tissue, organ, or organism into which a foreign polynucleic acid, such as a DNA vector or recombinant polynucleic acid molecule. A “transgenic” or “transformed” cell or organism also includes progeny of the cell or organism and progeny produced from a breeding program employing such a “transgenic” plant as a parent in a cross and exhibiting an altered phenotype resulting from the presence of the foreign polynucleic acid molecule.

It is also to be understood that two different transgenic plants can also be mated to produce offspring that contain two independently segregating added, exogenous genes. Selfing of appropriate progeny can produce plants that are homozygous for both added, exogenous genes. Back-crossing to a parental plant and out-crossing with a non-transgenic plant are also contemplated, as is vegetative propagation. Descriptions of other breeding methods that are commonly used for different traits and crops can be found in one of several references, e.g., Fehr, in Breeding Methods for Cultivar Development, Wilcox J. ed., American Society of Agronomy, Madison Wis. (1987).

A grass turfgrass stand is cultivated in private and public areas. A good turfgrass stand has both beauty and usefulness; its maintenance for golf, tennis, baseball, football, and other sports fields is a costly and specialized procedure. A turfgrass stand of the turfgrass of the present invention can be effectively managed for weed control by the application of a glyphosate containing herbicide. Turfgrasses are also used on along roadway right of ways and highway medians, reduced mowing and weed control would be a substantial benefit to the maintenance of these turfgrass areas. A turfgrass stand of the present invention preferably comprises transgenic glyphosate tolerant, dwarf turfgrass as a 50 percent or more component, more preferably a 75 percent component, and even more preferably greater than a 90 percent component. A turfgrass stand of the present invention has a growth rate of about 90 percent or less of a conventional turfgrass stand of the same genetic background, or about 75 percent or less of a conventional turfgrass stand of the same genetic background. A turfgrass stand of the present invention more preferably has a growth rate of about 50 percent or less of a conventional turfgrass stand of the same genetic background. A turfgrass stand of the present invention has a growth rate of about 25 percent or less of a conventional turfgrass stand of the same genetic background. A turfgrass stand of the present invention may have 10-90 percent of the growth rate of a conventional turfgrass stand of the same genetic background.

Gibberellic acid treatment temporally restores normal plant growth. For example, transgenic turfgrass seeds are coated with different concentrations of a commercial formulation of GA₃ (Release®, Abbott Labs, Abbott Park, Ill.). GA treatments to the seed, soil, and foliar application restores normal growth and development of turfgrass plants. Three methods of addition of GA₃ restores stature to the plants when seeds are sown and plants grown in the greenhouse and field. The GA₃ is added to seeds as Release® 10 SP (Abbott Labs) milled with talc powder. The GA₃ is also added as a one-step seed treatment with a suspension in water of Release® 10 SP, polyethylene glycol (3,000 to 20,000 MW) and talc powder. GA₃ concentrations between 5 and 20 ppm restore normal shoot height GA₃ treatments as a soil drench also restores seed emergence timing and plant height during early seedling growth. Rates of GA₃ between 1×10⁻⁶ and 1×10⁻⁵ M, when added to soil either immediately before planting or immediately after, restores normal shoot length. A foliar spray of GA₃ restores normal stature of plants. GA₃ restores normal vegetative development when sprayed on the foliage of GA-deficient dwarf plants during vegetative development at rates between 10⁻⁴ and 10⁻⁶ M plus a surfactant, for example, Tween 20, 0.05% v/v. Other rates and timing of applications can be tested to determine an effective amount of a gibberellic acid to apply to provide the desired level of growth. Other commercially available gibberellic acid formulations are expected to provide similar temporary restoration of vegetative growth. Gibberellic acid is also known to be a component of flower formation, in certain extreme dwarf phenotypes of the present invention it may be necessary to provide exogenous treatment of GA formulations to induce flower formation inorder to provide flowers for seed production or for conventional breeding.

Unless otherwise noted, terms are to be understood according to conventional usage by those of ordinary skill in the relevant art. Definitions of common terms in molecular biology may also be found in Rieger et al., Glossary of Genetics: Classical and Molecular, 5th edition, Springer-Verlag: New York, 1991; and Lewin, Genes V, Oxford University Press: New York, 1994. The nomenclature for DNA bases as set forth at 37 CFR §1.822 is used.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### Example 1

The DNA constructs of the present invention contain two plant expression cassettes, a first cassette provides for the expression of a dwarfing gene product that comprises a promoter that functions in plants operably linked to a GA 2-oxidase coding sequence, operably linked to a 3′ untranslated region. A second cassette is contained in the DNA construct, this cassette provides for the expression of a herbicide tolerance gene product that comprises a promoter that functions in plants operably linked to a glyphosate resistant EPSPS coding sequence, operably linked to a 3′ untranslated region. The methods used to assemble DNA fragments into operably linked elements are well known in the art of recombinant DNA (Sambrook et al, 1989).

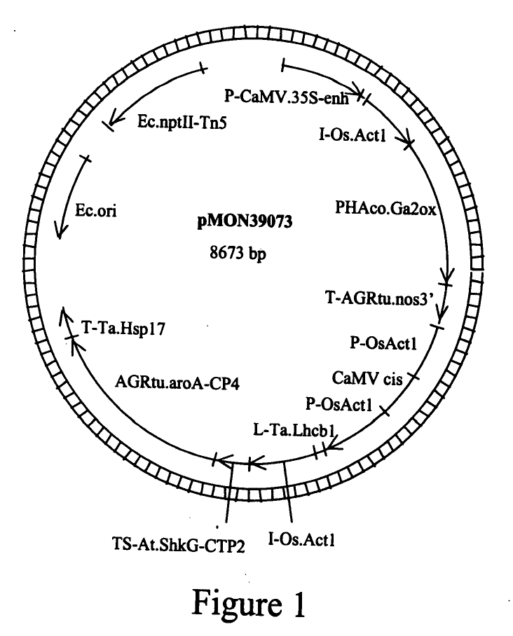

The DNA construct, pMON39073 (FIG. 1) is a plasmid that contains the maintenance elements of the plasmid backbone, such as, an origin of replication (Ec.ori) and a bacterial selectable marker gene (Ec.nptII-Tn5). The plasmid backbone is not a critical part of the invention, any suitable plasmid that allows for the maintenance of the plasmid in a bacterial cell and selection of the bacteria containing such plasmid is sufficient. The plant expression cassettes of pMON39073 are in a 5′-3′ orientation: the first cassette provides expression of a GA 2-oxidase that comprises a cauliflower mosaic virus 35S promoter with duplicated enhancer (P-CaMV.35S-enh, U.S. Pat. No. 5,322,938) linked to the rice actin 1 intron (I-Os.Act1, U.S. Pat. No. 5,641,876) linked to the GA 2-oxidase coding sequence from bean (PHAco.Ga2ox, U.S. Pat. No. 6,670,527) linked to the nopaline synthase transcriptional terminator (T-AGRtu.nos3′, also referred to as NOS 3′, Fraley et al. Proc. Natl. Acad. Sci. USA 80:4803-4807, 1983). This first cassette is also linked to a second plant expression cassette in a 5′-3′ orientation that provides expression of a glyphosate resistant EPSPS that comprises a first promoter fragment from rice actin 1 promoter (U.S. Pat. No. 5,641,876) linked to a CaMV cis element, linked to a second promoter fragment from rice actin 1 promoter, linked to a wheat CAB 5′ leader (L-Ta.LHcb1, WO0011200A2), linked to the rice actin 1 intron, linked to a chloroplast transit peptide (TS-At.ShkG-CTP2, also referred to as CTP2, Klee et al., Mol. Gen. Genet. 210:47-442, 1987), linked to a glyphosate resistant EPSPS (AGRtu.aroA-CP4, also referred to a EPSPS-CP4 or CP4 EPSPS, U.S. Pat. No. 5,633,435), linked to a wheat heat shock 17 3′ termination region (T-Ta.Hsp17, WO0011200A2). An isolated linear DNA fragment comprising the two plant expression cassettes is purified and used to coat gold in the particle bombardment transformation method.

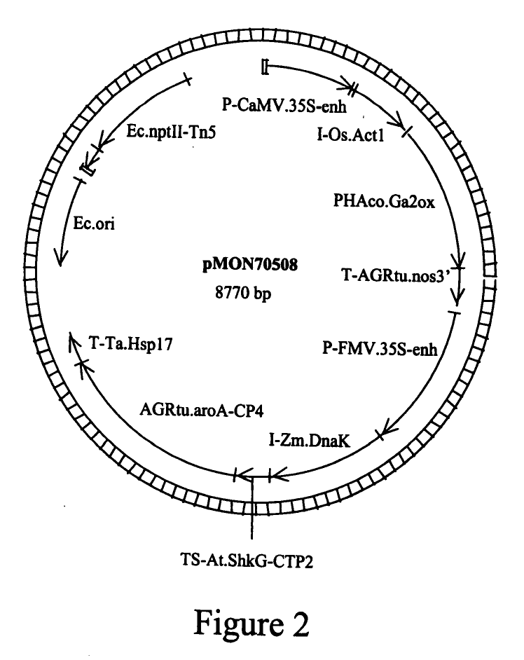

The DNA construct, pMON70508 (FIG. 2) contains two plant expression cassettes, the first cassette is the GA 2-oxidase expression cassette identical to that described in pMON39073. The second cassette provides a glyphosate tolerance gene comprising a figwort mosaic virus 35S promoter that has been duplicated (P-FMV.35S-enh, U.S. Pat. No. 6,018,100) linked to a maize heat shock 70 intron (I-Zm.DnaK, U.S. Pat. No. 5,424,412), linked to EPSPS-CP4, and linked to the T-Ta.Hsp17 termination region. An isolated linear DNA fragment comprising the two plant expression cassettes is purified and used to coat gold particles for use in the particle bombardment transformation method.

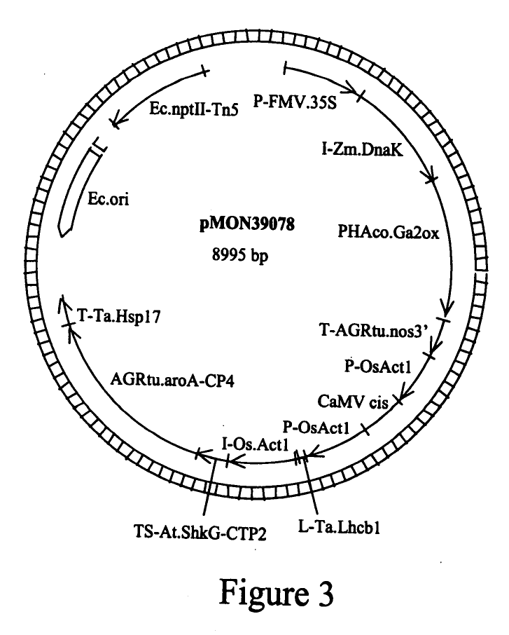

The DNA construct, pMON39078 (FIG. 3) contains two plant expression cassettes, the first cassette is the GA 2-oxidase expression cassette that comprises a single figwort mosaic virus 35S promoter (P-FMV.35S, U.S. Pat. No. 6,018,100), linked to I-Zm.DnaK, linked to PHAco.Ga2ox, and linked to T-AGRtu.nos3′. The first cassette is linked to a second cassette that provides expression of EPSPS-CP4 identical to that described in pMON39073. An isolated linear DNA fragment comprising the two plant expression cassettes is purified and used to coat gold particles for use in the particle bombardment transformation method.

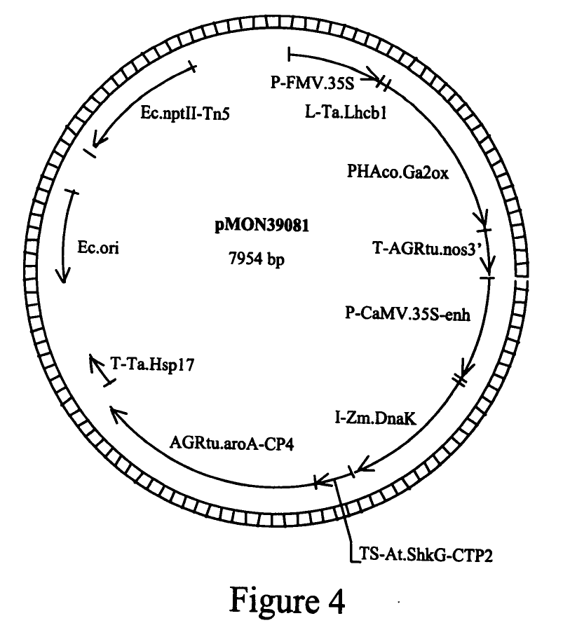

The DNA construct, pMON39081 (FIG. 4) contains two plant expression cassettes, the first cassette is the GA 2-oxidase expression cassette that comprises a single figwort mosaic virus 35S promoter (P-FMV.35S), linked to the L-Ta.Lhcb1 leader, linked to PHAco.Ga2ox, and linked to T-AGRtu.nos3′. The first cassette is linked to a second cassette that provides expression of EPSPS-CP4, the promoter being the P-CaMV.35S.enh promoter linked to I-Zm.DnaK, linked to AGRtu.aroA-CP4, and linked to T-Ta.Hsp17. An isolated linear DNA fragment comprising the two plant expression cassettes is purified and used to coat gold particles for use in the particle bombardment transformation method.

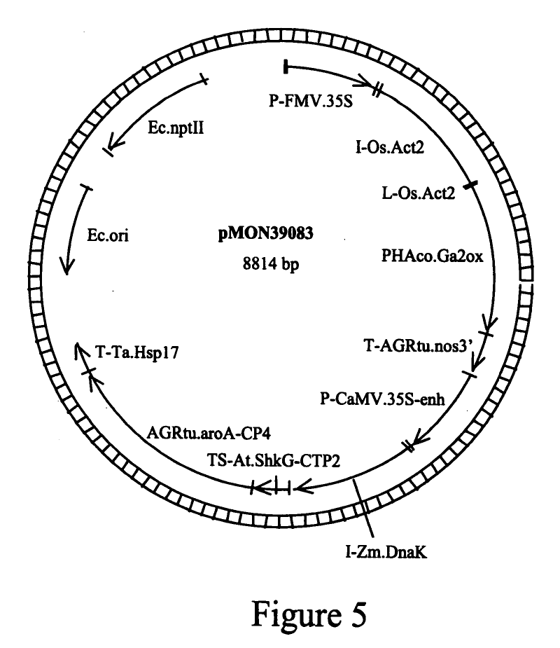

The DNA construct, pMON39083 (FIG. 5) contains two plant expression cassettes, the first cassette is the GA 2-oxidase expression cassette that comprises a single figwort mosaic virus 35S promoter (P-FMV.35S), linked to rice actin 2 intron (I-Os.Act2, U.S. Pat. No. 6,429,357) and rice actin 2 leader (L-Os.Act2, U.S. Pat. No. 6,429,357), linked to PHAco.Ga2ox, and linked to T-AGRtu.nos3′. The first cassette is linked to a second cassette that provides expression of EPSPS-CP4 identical to the cassette described for pMON39081. An isolated linear DNA fragment comprising the two plant expression cassettes is purified and used to coat gold particles for use in the particle bombardment transformation method.

Other promoters, such as, the Zea mays PPDK promoter for leaf expression or the nopaline synthase promoter for low constitutive expression of the GA 2-oxidase coding sequence are useful to provide various levels of control of the dwarf phenotype.

### Example 2

Description of Turfgrass Transformation

Creeping bentgrass and Kentucky bluegrass was transformed with the DNA constructs of the present invention containing the herbicide tolerance gene and the dwarfing gene. The bentgrass and bluegrass recipient cells used for the transformation were derived embryogenic callus cultures created from surface sterilized mature turfgrass seeds (Zhong et al. Plant Cell Rep. 10:453-456). Embryogenic callus was induced on callus initiation medium that comprises MS salts (Murashige and Skoog, Physiol. Plant. 15:473-497, 1962) and vitamins, 3% sucrose, 500 mg/L casein hydrolysate, 6.6 mg/L dicamba, and 0.5 mg/L 6-BAP and 0.2% gelgro as gelling agent. Approximately 4 to 6 weeks after callus initiation, embryogenic callus cultures were selected and maintained as culture lines by routine transferring of the callus cultures to fresh medium every 4 weeks.

The DNA construct was introduced into the embyogenic callus cultures via a particle bombardment process. In general, embryogenic callus cultures are pretreated with maintenance medium (Zhong et al. Plant Cell Rep. 10:453-456) with the addition of 0.2-0.3 M mannitol and 0.2-0.3 M sorbitol for 4 hours to 16 hours. Gold particles were coated with DNA, then the coated DNA microprojectiles were bombarded into embryogenic callus cultures using a gene gun. Selection of transgenic cells was initiated with either 1 or 2 mM glyphosate for 3-4 weeks, then raised to 2 or 3 mM glyphosate for 3-4 weeks. After 6-8 weeks on selection, shoots were regenerated in the presence of 0.1 mM glyphosate. Regenerated plantlets were transplanted to soil to greenhouse. After the plantlets were established in the greenhouse they were then treated with Roundup® herbicide to confirm the herbicide resistance in these transgenic plants

St Augustinegrass recipient cells are transformed with the DNA constructs of the present invention by using St. Augustinegrass inflorescence-derived embryogenic callus. The callus is maintained in the dark at 24-28° C. on F1DG medium [MSO medium (Table 1)+1 mg/L 2,4-D, 0.5 g/L MES buffer, 0.5 g/L casein hydrolysate, 1.5 g/L proline] and transferred to fresh medium approximately monthly. Approximately 0.2-0.3 g of callus tissue (each callus is about 2 mm) selected for transformation by particle bombardment are transferred to filter paper on F1DG or MS1DG medium (MSO medium+1 mg/L 2,4-D). Prior to bombardment, the calli are plasmolyzed for 4-6 hours to overnight on MS1DG medium supplemented with osmoticum (0.25 M mannitol+0.25 M sorbitol). The calli are bombarded 3 times at 900 psi using a Biolistic™ PDS-1000/He, with microprojectiles coated with an isolated linear fragment containing the plant expression cassettes of DNA constructs pMON39073, pMON3908 1, pMON70508, pMON39089, or pMON39091. The bombarded calli are transferred twenty-four hours later to F1DG medium, and at about six days the calli are transferred MS1DG medium containing 0.2-0.5 mM glyphosate.

The calli are maintained six weeks in the dark, after which time all surviving embryogenic sectors are transferred to regeneration medium (MSO medium+2 mg/L benzyladenine) containing glyphosate, ranging from 0.05 to 0.10 mM. The calli are maintained on this medium for five (5) weeks. At the end of the third (3) week, calli produce etiolated shoots and shoot buds, and are moved into illumination having a sixteen (16) hour light, eight (8) hour dark photoperiod. At the end of the fifth (5) week on SAR/glyphosate medium, calli are moved to MSO medium containing 0.02 mM glyphosate. Transgenic shoots are identified as darker-green, with healthy roots present in the medium.

TABLE 1
MSO Medium Composition¹

| Component | Weight (per liter) | |
|---|---|---|
| MS Salts | 1 | X |
| MS Vitamins | 1 | X |
| Sucrose | 30 | g |
| Gelrite | 3.0 | g |

¹pH adjusted to 5.8

Shoots from potentially transgenic plantlets are subjected to testing for the expression of the CP4EPSPS protein, using an indicator strip (RUR-HS Test Kit, Strategic Diagnostics, Inc., Newark, Del.). Shoots showing positive are maintained on MSO+0.01 mM glyphosate until they are larger and producing a healthy root system, after which they are moved to soil in the greenhouse. The transgenic turf grass cells, leaves, pollen, seeds, roots, rhizomes, or other parts containing plant cells transformed with DNA constructs that provide glyphosate tolerance and dwarf growth phenotype are an aspect of the present invention.

### Example 3

Glyphosate tolerance is tested on greenhouse grown transgenic turfgrass lines as follows: The R₀ transformant plant is grown in soil to where the roots reach the bottom of a 4″ square pot. The plant is then sprayed with Roundup® at 32 oz/acre. After 4 weeks, plants are scored numerically (1-5 scale) for Roundup® survival: 1 for dead, 2 for severe damage and dying, 3 for stunted or deformed regrowth and/or survival with considerable damage, 4 for minimal damage and recovering with normal regrowth, and 5 for undamaged. Lines that survived glyphosate spray were rated for dwarf phenotype (intermediate or extreme). Bentgrass, for example, transformed with pMON39073 showed thirty-one percent of the transgenic lines with extreme dwarf phenotype, fify-three percent of the lines with intermediate dwarf phenotype, and sixteen percent of the lines with normal growth phenotype. Table 2 shows the numbers of transgenic bluegrass lines produced from transformation with the various DNA constructs of the present invention, the number of lines that were treated with glyphosate and those that survived, and the number that show an intermediate or extreme dwarf phenotype. Table 3 shows the same analysis with transgenic St Augustinegrass.

TABLE 2
Bluegrass glyphosate treatment and dwarf phenotypes

| | | | # lines with | # lines extreme |
|---|---|---|---|---|
| | | # survived/ | intermediate | dwarf |
| Plasmid | # lines | # sprayed | dwarf phenotype | phenotype |
| pMON39073 | 59 | 57/58 | 19 (33%) | 6 (11%) |
| pMON70508 | 145 | 137/143 | 53 (37%) | 31 (22%) |
| pMON39083 | 98 | 44/45 | 21 (47%) | 11 (24%) |

TABLE 3
St Augustinegrass glyphosate treatment and dwarf phenotypes

| | | | # lines with | # lines extreme |
|---|---|---|---|---|
| | | # survived/ | intermediate | dwarf |
| Plasmid | # lines | # sprayed | dwarf phenotype | phenotype |
| pMON39073 | 35 | 25/30 | 35% | 61% |
| pMON70508 | 171 | 73/130 | 43% | 49% |
| pMON39078 | 224 | 47/65 | 31% | 47% |
| pMON39081 | 100 | 13/21 | 38% | 38% |

The glyphosate tolerance is demonstrated in field tests by treatment with 5% Roundup® Pro (glyphosate containing herbicide formulation) sprayed with a hand sprayer or an amount equivalent to 128 ounces Roundup® Pro per acre. The standard recommended rate is 1.25 to 2.5% Roundup® Pro or amount equivalent to 32 to 64 ounces Roundup® Pro per acre. Three applications of the glyphosate containing herbicide formulation are applied during the growing season, early summary, mid-summer and early fall at multiple locations. Vegetative injury is rated 2-4 weeks after treatment. Glyphosate can induce male fertility in transgenic plants where the expression of the glyphosate resistant enzyme in the male reproductive tissue is insufficient to provide glyphosate tolerance. Transgenic turfgrass lines can be selected from a population of lines that are vegetative glyphosate tolerant but reproductively sensitive. When these lines are treated with glyphosate prior to flowering, pollen formation will be inhibited resulting in male sterile plants. This trait is an advantage in turfgrass to limit pollen production and potential outcrossing.

### Example 4

The growth rate and dwarf severity phenotype are assayed for the various transgenic turfgrass species. Dwarf phenotypes are scored for plants that show a Roundup® survival score of 4-5. Node-cuttings are taken from the transgenic plants as well as reference plants (including the wild-type progenitor, and a naturally-occurring dwarf line 80-10), and rooted in soil in 4″ pots. After cuttings are rooted, the pots are placed under strong light and spaced evenly apart After 3-4 weeks, internode lengths of the longest stolon are measured, and internodes are numbered starting at the base of the stolon. Furthermore, the longest fully expanded leaf at the end of each internode is measured. These measurements are compared to that of reference lines.

Foliar growth rate is determined by cutting control plants and transgenic to a uniform height (10 cm). The growth rate (percent increase) is determined by measuring the height (cm) at one week and two weeks after cutting. As illustrated in FIG. 4, four transgenic bluegrass lines that have intermediate and extreme dwarf phenotypes were assayed for growth rate. The transgenic lines range from 25% to 105% increase in height at the two week time point, the control nontransgenic grass increased in height 120%-125%. The relative increase in height of the transgenic lines compared to the control lines ranged from about 20% (25%/120%) for the most extreme dwarf line Bx01-5609, to about 87% (105%/120%) for the Tx01-2862 line.

TABLE 4
Growth of transgenic and control bluegrass

| | Plant height (cm) | | | | dwarf |
|---|---|---|---|---|---|
| | 0 week | 1 week | 2 weeks | | phenotype |
| Control | 10 | 15.5 | 22 | (120%) | |
| Tx01-2900 | 10 | 13 | 16 | (60%) | intermediate |
| Tx01-2862 | 10 | 14.5 | 20.5 | (105%) | intermediate |
| Tx01-2875 | 10 | 13.5 | 17.5 | (75%) | intermediate |
| Control | 10 | 17.5 | 22.5 | (125%) | |
| Bx01-5609 | 10 | 10.5 | 12.5 | (25%) | extreme |

It was observed that the transgenic lines produced more tillers that the nontransgenic controls. For example, line Tx01-2900 was able to generate 15 units of single tiller plants as compared to a nontransgenic control that only generated 6-8 units of single tiller plants during the same period of time. More tillers produces a thicker, fuller more dense lawn. A lawn density is rated on a 0-9 scale, the transgenic turfgrass plants of the present invention provide for a turfgrass stand that has a density rating of greater that six, preferably greater than seven and more preferably greater than eight. Additionally, for the turfgrasses that are sold as plugs or sod, the ability of these plants to produce more vegetative tillers will speed up the vegetative reproduction of units of turfgrass for sale.

Field tests of transgenic bluegrass lines were conducted that compared various bluegrass genotypes at various locations (Wisconsin, WI and Alabama, Al) to determine the relative growth phenotype of the transgenic lines. The results shown in Table 5 demonstrate that Bx01-5609, consistently has a reduced growth height compared to all other genotypes tested. The transformed heat tolerant bluegrass lines, Tx01-2862, Tx01-2875 and Tx01-2900 show reduced plant height when compared to HB130, the line from which they were derived and HB 129. The transgenic lines were similar to a number of conventionally bred Kentucky bluegrasses (Unique, Limousine, Midnight), and HB 329 (dwarf, heat tolerant bluegrass) that have a similar low growth habit. These results confirm that the individual transformed plants have reduced growth when compared to their parental genotype and non-transformed tissue culture lines. Percentage plot coverage was measured using a percent occupancy measure (percentage of grids in the plot with green tissue present).

TABLE 5
Mown competitive Trials - Plant height and percent plot
coverage measures.

| | Plant height (cm) | | | | Percent plot coverage | | | |
|---|---|---|---|---|---|---|---|---|
| Line | WI | | AL | | WI | | AL | |
| Bx01-5609 | 4.3 | d | 4.3 | g | 2.3 | cd | 3.8 | d |
| Tx01-2875 | 5.3 | cd | 6.3 | c-f | 2.9 | a | 5.8 | a |
| HB-329 | 5.4 | cd | 6.5 | c-f | 2.9 | a | 5.3 | abc |
| TX01-2862 | 5.4 | cd | 7.0 | a-d | 2.9 | a | 5.3 | abc |
| Unique | 5.4 | cd | 5.5 | f | 2.8 | ab | 4.3 | bcd |
| 010607C Unique | 5.6 | cd | 6.8 | b-e | 2.7 | abc | 5.3 | abc |
| Limousine | 5.6 | cd | 6.5 | c-f | 2.7 | abc | 4.5 | a-d |
| South Dakota | 5.7 | cd | 7.8 | ab | 2.4 | bcd | 5.5 | ab |
| Ascot | 6.2 | bcd | 7.0 | a-d | 2.6 | abc | 4.8 | a-d |
| HB-129 | 6.2 | bcd | 8.0 | a | 2.9 | a | 5.3 | abc |
| 021128C HB-130 | 6.3 | bc | 7.3 | abc | 2.9 | a | 5.8 | a |
| Tx01-2900 | 6.3 | bc | 6.0 | def | 2.9 | a | 4.3 | bcd |
| 02080S HB-130 | 6.5 | bc | 7.0 | a-d | 2.9 | a | 5.5 | ab |
| Abbey | 6.9 | abc | 6.5 | c-f | 2.5 | a-d | 5.3 | abc |
| HB-130 | 6.9 | abc | 8.0 | a | 2.9 | a | 5.8 | a |
| Midnight | 6.9 | abc | 6.3 | c-f | 2.6 | abc | 5.5 | ab |
| Texas bluegrass | 7.8 | ab | 5.8 | ef | 2.1 | d | 4.0 | cd |
| Touchdown | 8.4 | a | 6.8 | b-e | 2.7 | abc | 5.0 | a-d |
| LSD (.05) | 1.9 | | 1.9 | | 0.5 | | 1.3 | |

Values within columns with the same letters are not significantly different.

Rhizome growth was measured by the appearance of tillers away from the central crown of the plant. Plants were grown in Alabama (Al), Oregon (OR), and California (CA) the results are shown in Table 6. In general the most extreme dwarf line, BX01-5609, showed the most reduction in mean rhizome length. The Tx01 lines (2875, 2862, 2900) did not show a significant difference in mean rhizome length compared to HB 130. This indicates that transgenic lines can be selected that have a significantly reduced spread phenotype as well as lines that have reduced height, but have retained their ability to spread horizontally within their growth environment.

TABLE 6
Mean rhizome length (cm) at three locations

| Line | AL | | OR | | CA | |
|---|---|---|---|---|---|---|
| Bx01-5609 | 5.8 | g | 0.6 | e | 0.5 | e |
| Midnight | 8.5 | b-f | 10.2 | a-d | 8.3 | a-d |
| 010607C Unique | 8.3 | b-g | 9.2 | bcd | 6.5 | cd |
| Limousine | 6.7 | fg | 10.2 | a-d | 9.6 | abc |
| Unique | 10.7 | abc | 7.2 | d | 7.7 | a-d |
| Ascot | 7.6 | d-g | 11.3 | abc | 9.2 | abc |
| HB-329 | 9.3 | b-f | 9.2 | bcd | 8.3 | a-d |
| South Dakota | 10.3 | abc | 10.2 | a-d | 5.5 | d |
| Tx01-2875 | 9.9 | a-e | 9.2 | bcd | 9.0 | abc |
| 021128C HB-130 | 12.1 | a | 11.7 | ab | 10.6 | a |
| Texas bluegrass | 7.4 | efg | 10.3 | a-d | 1.3 | e |
| Touchdown | 9.0 | b-f | 8.2 | cd | 6.8 | bcd |
| TX01-2862 | 8.1 | c-g | 7.6 | d | 8.0 | a-d |
| Tx01-2900 | 10.1 | a-d | 8.4 | cd | 8.6 | a-d |
| 02080S HB-130 | 9.9 | a-e | 11.0 | abc | 9.9 | ab |
| Abbey | 10.8 | ab | 13.3 | a | 7.6 | a-d |
| HB-129 | 9.8 | a-e | 11.2 | abc | 9.1 | abc |
| HB-130 | 10.4 | abc | 11.3 | abc | 8.7 | a-d |
| LSD (.05) | 2.6 | | 3.3 | | 3.2 | |

Values within columns with the same letters are not significantly different.

Transgenic bluegrass of the present invention can also show reduced fertility as a result of reduced gibberellin in the plant during flower development. Table 7 shows the percent pollen germination of transgenic lines Tx01-2900, Tx01-2875, Tx01-2862, and Bx01-5609. The most dwarfed line, Bx01-5609, did not produce anthers and hence no pollen. The fertility of dwarf lines is generally correlated to the severity of the dwarf phenotype. The reduced fertility is a useful trait for a transgenic turf grass. Reduced or no pollen production means reduced opportunities for outcrossing to nontransgenic grass species. Grass pollen is an allergen too many people, having a turfgrass that produces less pollen would be advantageous to reduce the allergen load in the atmosphere. Also, in other tests, the severe dwarf lines had a very small amount seed production, this would severely limit any substantial spread of the grass by seed.

TABLE 7
Comparison of percent pollen germination among transgenic and
conventional bluegrass lines.

Percent pollen germination

| Entry | Rep 1 | Rep 2 | Rep 3 | Mean |
|---|---|---|---|---|
| Touchdown | 16.4 | 18.2 | 30.7 | 21.8 |
| Tx01-2900 | 26.4 | 12.3 | 22.9 | 20.5 |
| HB 130 | 13.1 | 23.3 | 19.3 | 18.6 |
| 021128c HB 130 | 23.8 | 15.3 | 11.3 | 16.8 |
| Tx01-2875 | 17.3 | 12.8 | 14.3 | 14.8 |
| 02080S HB 130 | 17.2 | 14.1 | 11.3 | 14.2 |
| HB 329 | 15.0 | 17.8 | 7.4 | 13.4 |
| South Dakota | 10.0 | 7.8 | 19.9 | 12.6 |
| 010607c Unique | 16.8 | 2.0 | 14.5 | 11.1 |
| Tx01-2862 | 15.1 | 11.3 | 6.9 | 11.1 |
| Unique | 13.8 | 3.3 | 6.7 | 7.9 |
| Bx01-5609 | *none | none | none | none |
| HB 129 | 11.4 | | 2.0 | 6.7 |

*no anthers produced

Having illustrated and described the principles of the present invention, it should be apparent to persons skilled in the art that the invention can be modified in arrangement and detail without departing from such principles. We claim all modifications that are within the spirit and scope of the appended claims.

All publications and published patent documents cited in this specification are incorporated herein by reference to the same extent as if each individual publication or patent application was specifically and individually indicated to be incorporated by reference.

We claim:

1. A method for producing a low maintenance turfgrass plant comprising the steps of: a) contacting a recipient turfgrass plant cell with a transgene DNA construct comprising a herbicide tolerance gene and a heterologous dwarfing gene, wherein said DNA construct is incorporated into the genome of the recipient turfgrass plant cell; and b) regenerating the recipient plant cell into a turfgrass plant; wherein said turfgrass plant is herbicide tolerant and has a dwarf growth phenotype.

2. The method of claim 1, wherein said herbicide tolerance gene is a glyphosate tolerance gene.

3. The method of claim 2, wherein said glyphosate tolerance gene comprises a glyphosate resistant 5-enolpyruvyl-3-phosphoshikimate synthase coding sequence.

4. The method of claim 1, wherein said dwarfing gene is a gibberellic acid level reducing gene.

5. The method of claim 4, wherein said gibberellic acid level reducing gene comprises a gibberellin 2-oxidase coding sequence.

6. The method of claim 1, wherein said turfgrass plant is selected from the group consisting of bentgrass, St Augustinegrass, and bluegrass.

7. The method of claim 1, wherein said transgene DNA construct comprises a first plant expression cassette comprising a constitutive promoter that functions in said turfgrass plant cell operably linked to a DNA molecule that encodes a glyphosate resistant enzyme, and linked to a second plant expression cassette comprising a promoter that functions in turfgrass plant cells operably linked to a DNA molecule that encodes a gibberellin 2-oxidase enzyme.

8. A method for controlling weeds in a turfgrass stand comprising: applying an effective amount of a glyphosate containing herbicide formulation to said turfgrass stand, wherein said turfgrass stand comprises a transgenic grass comprising a glyphosate tolerance transgene and a dwarfing transgene.

9. A method for inducing growth of a transgenic herbicide tolerant, dwarf turfgrass comprising: applying an effective amount a bioactive gibberellic acid containing formulation to said turfgrass seed, roots, or foliage.

10. A transgenic bluegrass plant comprising a transgene comprising a heterologous DNA molecule that provides herbicide tolerance and dwarf growth phenotype;

11. The transgenic bluegrass plant of claim 10, wherein said heterologous DNA molecule expresses a glyphosate resistant 5-enolpyruvyl-3-phosphoshikimate synthase.

12. The transgenic bluegrass plant of claim 10, wherein said heterologous DNA molecule expresses a gibberellin 2-oxidase.

13. The transgenic bluegrass plant of claim 10, wherein said bluegrass plant is between 25 percent and 40 percent of the height of a same bluegrass variety not containing the heterologous DNA molecule.

14. The transgenic bluegrass plant of claim 10, wherein said bluegrass plant is between 41 percent and 55 percent of the height of a same bluegrass variety not containing the heterologous DNA molecule.

15. The transgenic bluegrass plant of claim 10, wherein said bluegrass plant is between 56 percent and 70 percent of the height of a same bluegrass variety not containing the heterologous DNA molecule.

16. The transgenic bluegrass plant of claim 10, wherein said bluegrass plant is between 71 percent and 85 percent of the height of a same bluegrass variety not containing the heterologous DNA molecule.

17. The transgenic bluegrass plant of claim 10, wherein said bluegrass plant has reduced pollen production compared to a same bluegrass variety not containing the heterologous DNA molecule.

18. The transgenic bluegrass plant of claim 10, wherein said bluegrass plant has a reduced height and no significantly different rhizome length compared to a same bluegrass variety not containing the heterologous DNA molecule.

19. The transgenic bluegrass plant of claim 10, further comprising a turfgrass stand of said bluegrass plant, wherein said stand has a higher lawn density rating compared to a same bluegrass plant variety not containing the heterologous DNA molecule.

20. The turfgrass stand of claim 19, wherein said stand requires less mowing during a growing season compared to a stand of a same bluegrass plant variety not containing the heterologous DNA molecule.
